(19) United States
(12) Patent Application Publication
(10) Pub. No.: US 2022/0241207 A1
(43) Pub. Date: Aug. 4, 2022

(54) EXTENDED-RELEASE SOLID ORAL DOSAGE FORM COMPRISING VITAMIN B12 AND A VITAMIN B12 DEPLETING DRUG

(71) Applicant: DSM IP ASSETS B.V., Heerlen (NL)

(72) Inventors: Martin Thomas KUENTZ, Kaiseraugst (CH); Zdravka MISIC, Kaiseraugst (CH); Andreas NIEDERQUELL, Kaiseraugst (CH); Ralph SCHNEITER, Kaiseraugst (CH)

(21) Appl. No.: 17/619,464

(22) PCT Filed: Jun. 17, 2020
(86) PCT No.: PCT/EP2020/066757

§ 371 Date: Dec. 15, 2021

(30) Foreign Application Priority Data
Jun. 17, 2019 (EP) 19180447.5

(51) Int. Cl. A61K 9/24; A61K 31/7048; A61K 31/714; A61K 31/155; A61K 9/20

(52) U.S. Cl. CPC A61K 9/209; A61K 31/7048; A61K 9/2013; A61K 31/155; A61K 31/714

(57) ABSTRACT

The present invention relates to a solid oral dosage form comprising an extended-release tablet core that is coated with an immediate-release coating, wherein said extended-release tablet core comprises at least one vitamin B12 depleting drug, and wherein said immediate-release coating comprises vitamin B12 or a spray dried formulation thereof. In a preferred embodiment, the vitamin B12 depleting drug is metformin or a pharmaceutically acceptable salt thereof.

### BACKGROUND OF THE INVENTION

During long-term use of certain drugs, the serum vitamin B12 level decreases. An example of such a drug is metformin. Other medications are also known to deplete vitamin B12. Therefore, if a patient has had a low vitamin B12 level before, doctors are advised to monitor the patient's vitamin B12 level during the treatment (e.g. by doing blood tests).

Metformin is commercially available as immediate-release formulation, as extended-release formulation and also as fixed-dose combination (FDC).

An example of a combination product is SYNJARDY® XR. Each film-coated tablet of SYNJARDY® XR comprises an extended-release metformin hydrochloride tablet core that is coated with the immediate-release drug substance empagliflozin. According to the package leaflet, a possible side effect of SYNJARDY® XR is a low vitamin B12 level or even vitamin B12 deficiency.

A lower than normal absorption of vitamin B12 can be compensated by an increased intake of vitamin B12. Some food (such as liver and kidney) comprises a relatively high amount of vitamin B12. However, for many patients and in particular for vegetarians and vegans, an increased consumption of edible offal is not an option. As an alternative, a vitamin B12 dietary supplement could be taken.

Whereas dietary supplements are easily accessible, it is known that patient compliance is notoriously low when two tablets instead of only one needs to be taken on a regular basis. This is particularly true for elderly diabetes patients as there is often further co-medication in diabetes patients. Co-medication increases the so-called “pill count” (i.e. number of tablets to be administered) and hence the risk of poor adherence to therapy. Patient compliance becomes even worse if one tablet comprises a prescription drug (Rx), whereas the second tablet may be bought in the supermarket. So far, patients are just not used to the combined intake of tablets that originate from very different supply channels. In addition, symptoms from vitamin B12 deficiency occur rather slowly and may be indistinguishable from symptoms caused by chronically high blood sugar and diabetes. Therefore, the omittance of one or two vitamin B12 supplements will not instantly worsen the patient's condition. This also negatively affects patient's compliance as the perception of not feeling much relief during the course of taking the medication influences the patient's decision to discontinue the treatment regimen.

There is a need for a better manner to deal with vitamin B12 depletion induced by an oral dosage form that comprises a vitamin B12 depleting drug.

### SUMMARY OF THE INVENTION

To reduce pill count and to increase patient compliance, vitamin B12 is added to an extended-release tablet that comprises a vitamin B12 depleting drug. Thereby, pill count reduction is achieved (i) by replacing an immediate-release formulation with an extended-release formulation and (ii) by including vitamin B12 into the formulation such that the additional intake of a vitamin B12 dietary supplement becomes superfluous.

The problems underlying the present invention are solved by a solid oral dosage form comprising an extended-release tablet core that is coated with an immediate-release coating, wherein said core comprises at least one vitamin B12 depleting drug, and wherein said coating comprises vitamin B12.

The solid oral dosage form releases vitamin B12 from the coating into the stomach of the patient, whereas the drug comprised in the tablet's extended-release core is mainly released in the patient's intestine. Due to this very specific release pattern, the vitamin B12 level in the patient's blood is increased or maintained in a meaningful manner.

Vitamin B12 can only be absorbed if released in the stomach. For absorption, vitamin B12 must form a complex with an intrinsic factor (B12-IF complex). The required intrinsic factor is present in the stomach but not in the intestine. If released after stomach (i.e. in the intestine), vitamin B12 is poorly absorbed or not absorbed at all.

To be released in the stomach, vitamin B12 should be included in the immediate-release coating of the dosage form of the invention. However, to be protected from damaging light, vitamin B12 should rather be placed within the core underneath the coating. Thus, a difficult choice has to be made between two alternatives that are equally undesirable.

Adding an overage of vitamin B12 and/or accepting an unfavorable short shelf life would be one way to solve this dilemma. However, the inventors have found more sophisticated technical solutions to protect vitamin B12 from light such that vitamin B12 can be included in the immediate-release coating despite of its light sensitivity.

In one embodiment, a spray dried formulation of vitamin B12 is included in the immediate-release coating of the invention. In comparison to vitamin B12 crystals, spray dried formulations of vitamin B12 have an increased light stability. Thus, in a preferred embodiment, the solid oral dosage form of the present invention comprises an extended-release tablet core that is coated with an immediate-release coating, wherein said core comprises at least one vitamin B12 depleting drug, and wherein said coating comprises a spray dried formulation of vitamin B12. The present invention also relates to the use of a spray dried formulation of vitamin B12 for manufacturing an immediate-release coating, wherein said immediate-release coating covers at least partially an extended-release tablet core which comprises at least one vitamin B12 depleting drug.

Alternatively, a second, protective layer can be added on the top of the coating which comprises vitamin B12. Such additional protective layer protects vitamin B12 from light and comprises optionally at least one light-protection agent such as titanium dioxide (TiO₂).

In the context of the present invention, the vitamin B12 depleting drug is preferably metformin HCl. Metformin HCl is a pharmaceutically acceptable salt of metformin and is mainly used in the treatment of type 2 diabetes. To treat type 2 diabetes even more effectively, metformin HCl might be combined with a second anti-diabetic drug such as empagliflozin.

To increase effectiveness and patient compliance, two drugs acting by different mechanisms can be combined in a fixed-dose combination (FDC). A preferred embodiment of the invention relates to an FDC of metformin HCl and at least one further anti-diabetic drug such as empagliflozin. Thus, the solid oral dosage form of the invention comprises preferably an extended-release tablet core that is coated with an immediate-release coating,
wherein said core comprises metformin HCl, and
wherein said coating comprises vitamin B12 or a spray dried formulation thereof and at least one further anti-diabetic drug being preferably empagliflozin.

To prevent or treat metformin induced vitamin B12 deficiency even more effectively, at least one source of ionic calcium can optionally be included in the extended-release core of the solid oral dosage form. Ionic calcium is obligatory for the B12-IF complex to attach to ileal cell surface receptors. Drugs such as metformin compete with calcium for the mucosal cell membrane, and therefore induce vitamin B12 malabsorption. Said vitamin B12 malabsorption is at least partially reversible with ionic calcium.

Whereas vitamin B12 is to be released in the stomach (and must therefore be part of the immediate-release coating of the dosage form), the source of ionic calcium may be located anywhere in the dosage form (e.g. within the coating and/or within the core). However, because most sources of ionic calcium require a considerable amount of space, at least one source of calcium ion is preferably included in the extended-release core of the solid oral dosage form. Thus, the solid oral dosage form of the invention comprises preferably an extended-release tablet core that is coated with an immediate-release coating,
wherein said core comprises metformin or a pharmaceutically acceptable salt thereof and at least one source of ionic calcium, and
wherein said coating comprises vitamin B12.

As a source of ionic calcium, any pharmaceutically acceptable calcium salt can be used. However, if included in the extended-release tablet core, the calcium salt is preferably a calcium salt of citric acid. The extended-release tablet core reaches the patient's intestine at least partially undissolved and therefore, the calcium salt within the tablet core should be sufficiently soluble in intestinal fluid. Calcium salts of citric acid have a good or even excellent solubility in intestinal fluid. Thus, the solid oral dosage form of the invention comprises preferably an extended-release tablet core that is coated with an immediate-release coating,
wherein said core comprises metformin or a pharmaceutically acceptable salt thereof and calcium citrate, and
wherein said coating comprises vitamin B12 or a spray dried formulation thereof.

In an even more preferred embodiment, the solid oral dosage form of the invention comprises preferably an extended-release tablet core that is coated with an immediate-release coating,
wherein said core comprises metformin HCl and calcium citrate, and
wherein said coating comprises a spray dried formulation of vitamin B12 and at least one further anti-diabetic drug being preferably empagliflozin.

The present invention also relates to the use of the herein described solid oral dosage as a medicament, such as to the use in the treatment or prevention of metformin induced vitamin B12 deficiency.

Finally, the present invention also relates to a method of manufacturing a solid oral dosage form which comprises an extended-release tablet core and at least one vitamin B12 depleting drug, wherein said extended-release tablet core is provided with an immediate-release coating that comprises vitamin B12 or a spray dried formulation thereof.

### DETAILED DESCRIPTION OF THE INVENTION

The present invention relates to a solid oral dosage form that releases vitamin B12 in the stomach and that releases a vitamin B12 depleting drug over an extended period of time in the intestine. The vitamin B12 depleting drug is preferably metformin HCl.

### Definitions

The term “comprising” is an open term. Therefore, the herein described solid oral dosage form may comprise more than one coating. For example, it may comprise two coatings, wherein the inner coating comprises a source of vitamin B12 and wherein the outer coating is a protective layer which protects the vitamin B12 of the inner coating from light. Similarly, the herein described solid oral dosage may comprise more than one source of calcium ions. For example, it may comprise calcium citrate and anhydrous dicalcium phosphate. The latter is useful when granulating metformin HCl and may also increase the absorption of vitamin B12 by a patient being treated with metformin. Thus, anhydrous dicalcium phosphate may have a double functionality.

The term “solid oral pharmaceutical dosage form” refers to a dosage form such as a tablet, a granule, a capsule and a sachet. Preferably, the term refers to a tablet or to a granule that comprises an extended-release core which is coated with at least one immediate-release coating. Granules may be filled into empty capsule shells or into any other container such as a sachet. Most preferably, the term “solid oral pharmaceutical dosage form” refers to a tablet that comprises an extended-release tablet core which is coated with at least one immediate-release coating.

In the context of the present invention, a “coating” is preferably a film coating. Typically, the immediate-release coating of the invention comprises undissolved particles. Said particles may be vitamin B12 crystals or spray dried particles comprising vitamin B12. Thus, due to the red color of vitamin B12, the coating of the invention might comprise red dots.

In the context of the present invention, the term “extended-release” relates to a reduction of the dosing frequency. In the context of the present invention, “extended-release” refers to the vitamin B12 depleting drug and means that the at least one vitamin B12 depleting drug (e.g. metformin HCl) is slowly released in the body over an extended period of time such that the dosing frequency (and thus the pill count) can be reduced. An example of such product is SYNJARDY® XR which needs to be taken orally once daily. Other products for the same medical indication that also contain metformin HCl but that are not extended-release (such as GLUCOPHAGE®) need to be taken three or even four times a day. The extended-release tablet core of the present invention may or may not have a lag-time. For example, the release of the at least one vitamin B12 depleting drug may be delayed until the tablet has passed through the stomach and is then released in the intestine over an extended period of time. However, even if there is a lag-time, the solid oral dosage form of the invention is typically not enteric coated.

The “immediate-release coating” of the present invention comprises vitamin B12 and optionally at least one further active pharmaceutical ingredient (API) such as empagliflozin. Therefore, vitamin B12 and any further optionally API are released into the body when the coating is being dissolved or is broken down in any other manner. After oral administration of the solid dosage form of the invention, the coating is broken down by gastric juice without delay such that most (if not all) of the vitamin B12 and any further optionally API are released into the stomach before the remains of the administered solid oral dosage form will reach the intestine. In a preferred embodiment, the immediate-release coating of the invention enables the release of vitamin B12 and optional empagliflozin into the stomach.

The optional “protective coating” of the invention covers the immediate-release coating of the invention at least partially (i.e. is the outer coating). After oral administration of the solid dosage form of the invention, the protective coating is broken down by gastric juice without delay such that gastric juice gains quickly access to the immediate-release coating of the invention (i.e. to the inner coating). Thus, the protective coating of the invention is not an enteric coating. The main purpose of the protective coating of the invention is to protect the inner coating's vitamin B12 from light. Therefore, the protective coating comprises preferably at least one light-protection agent. Although not preferred, it may also comprise any other API such as empagliflozin.

The solid pharmaceutical dosage form of the invention comprises preferably microcrystalline cellulose (MCC). MCC is a well-known excipient prepared by acid hydrolysis of cellulose. On industrial scale, MCC is obtained by hydrolysis of wood and/or cotton cellulose using dilute mineral acids. The treated pulp is then rinsed and spray-dried with or without an additional process step such as milling. Numerous types of microcrystalline cellulose (MCC) are available on the market. In the context of the present invention, the term “microcrystalline cellulose” includes any type of microcrystalline cellulose consisting of partially depolymerized cellulose such as the excipients listed in Table 1 of T. Vehovec et al.: “Influence of different types of commercially available microcrystalline cellulose on degradation of perindopril erbumine and enalapril maleate in binary mixtures”, Acta Pharm. 62 (2012), page 518. Also included is silicified microcrystalline cellulose such as PROSOLVO® SMCC. In the context of the present invention, the term “silicified microcrystalline cellulose” refers to an excipient comprising microcrystalline cellulose (MCC) and silicon dioxide such as colloidal silicon dioxide (CSD).

Vitamin B12 is a well-known water-soluble vitamin. In the context of the present invention, the term “vitamin B12” refers to any vitamer of vitamin B12 and includes vitamin B12 derivatives and/or metabolites of vitamin B12. Preferably, however, the term “vitamin B12” refers to cyanocobalamin. Cyanocobalamin may be produced by fermentation using suitable microorganisms.

“Crystalline vitamin B1” comprises at least 98 weight-% vitamin B12, based on the total weight of the crystals. In one embodiment, the solid oral dosage form of the invention does not comprise any crystalline vitamin B12.

The solid oral dosage form of the invention of the invention may comprise at least one spray dried formulation of vitamin B12. The expression “spray dried formulation of vitamin B1” refers to a powder which is obtainable by spray drying of an aqueous solution that comprises vitamin B12 and at least one excipient, wherein said at least one excipient is preferably selected from the group consisting of sodium citrate, trisodium citrate, citric acid, maltodextrin citric acid and modified food starch. In a preferred embodiment of the invention, the expression “spray dried formulation of vitamin B12” refers to a powder which is obtainable by spray drying an aqueous solution which comprises cyanocobalamin and at least one excipient, wherein said at least one excipient is preferably selected from the group consisting of sodium citrate, trisodium citrate, citric acid, maltodextrin and modified food starch.

Vitamin B12 crystals have a vitamin B12 content of at least 98 weight-%, based on the total weight of the crystals. Due to the presence of at least one excipient, the spray dried formulation of vitamin B12 comprises less than 90 weight-% of vitamin B12, based on the total weight of the spray dried formulation. The exact concentration of vitamin B12 in the spray dried formulation of vitamin B12 depends on the amount of excipient in the spray dried formulation. Preferably, the spray dried formulation of vitamin B12 of the invention comprises 1 weight-% or less of vitamin B12, based on the total weight of the spray dried formulation. The person skilled in the art understands that spray dried formulations of vitamin B12 being free of vitamin B12 are excluded. Also preferably, the spray dried formulation of vitamin B12 of the invention is a water-soluble powder or a water-dispersible powder comprising 1 weight-% or less of cyanocobalamin, based on the total weight of the powder. The person skilled in the art understands that powders being free of vitamin B12 are excluded. In the most preferred embodiment of the invention, the expression “spray dried formulation of vitamin B12” refers to a powder which is obtainable by spray drying an aqueous solution which comprises cyanocobalamin and at least one excipient, wherein said excipient is preferably selected from the group consisting of sodium citrate, trisodium citrate, citric acid, maltodextrin and modified food starch, and wherein said powder comprises 1 weight-% or less of cyanocobalamin, based on the total weight of the powder. Again, the person skilled in the art understands that powders being free of vitamin B12 are excluded.

In the context of the present invention, the term“metformin” refers to metformin or to a pharmaceutically acceptable salt thereof. The probably best known pharmaceutically acceptable salt of metformin is metformin HCl. Therefore, in the most preferred embodiment of the invention, the term “metformin” refers to metformin HCl.

Metformin HCl has a poor compactability and flowability. Therefore, metformin HCl is preferably granulated before tableting. During such granulation process, metformin is transformed into free-flowing, essentially dust-free granules that are easy to compress. In the context of the present invention, the term “granulated metformin” refers to granules comprising metformin HCl and at least one pharmaceutically acceptable excipient. The excipient is not particularly restricted. However, good quality granulated metformin is achieved when following the teaching of EP 2 938 362 which is hereby incorporated by reference.

In the context of the present invention, the term “calcium salt” refers to any pharmaceutically acceptable calcium salt. Thus, the term includes calcium phosphate, calcium carbonate and calcium citrate. Calcium carbonate is a chemical compound with the formula CaCO₃. The term “calcium citrate” includes monocalcium citrate, dicalcium citrate and tricalcium citrate. Known tricalcium citrate salts include anhydrous calcium citrate (i.e. Ca₃ (C₆H₅O₇)₂) and tricalcium dicitrate tetrahydrate (i.e. [Ca₃ (C₆H₅O₇)₂(H₂O)₂].H₂O). The term “calcium phosphate” includes anhydrous calcium phosphate and hydrous calcium phosphate. Known are anhydrous calcium phosphates, anhydrous monocalcium phosphate (Ca(H₂PO₄)₂), anhydrous dicalcium phosphate (CaHPO₄) or anhydrous tricalcium phosphate (Ca₃(PO₄)₂). In the most preferred embodiment of the invention, the term calcium salt refers to anhydrous dicalcium phosphate (CaHPO₄).

Extended-Release Core

The solid oral dosage form of the invention comprises an extended-release core that is preferably an extended-release tablet core. The purpose of said core is the release of the at least one vitamin B12 depleting drug over an extended period of time such that the dosing frequency of the vitamin B12 depleting drug can be reduced.

The extended-release core may be manufactured by any known manufacturing method as long as it releases the at least one vitamin B12 depleting drug slowly in the body over an extended period of time. Thus, it can be manufactured by e.g. granulation, extrusion or compression. Preferably, the extended-release core of the invention is an extended-release tablet core that is manufactured by compression with a tablet press.

The extended-release core of the invention and in particular the extended-release tablet core of the invention comprises preferably at least one vitamin B12 depleting drug and at least one pharmaceutically acceptable excipient. More preferably, it comprises metformin or a pharmaceutically acceptable salt thereof and at least one pharmaceutically acceptable excipient. Most preferably, it comprises metformin HCl and at least one pharmaceutically acceptable excipient.

Preferred pharmaceutically acceptable excipients are controlled release agents (such as hydroxypropyl methylcellulose), diluents (such as microcrystalline cellulose and silicified microcrystalline cellulose), lubricants (such as magnesium stearate and glyceryl behenate) and calcium salts. A particularly preferred pharmaceutically acceptable excipient is Avicel® DG which is known to be a combination of 75 weight-% microcrystalline cellulose and 25 weight-% anhydrous dicalcium phosphate.

In a preferred embodiment of the invention, the extended-release tablet core of the invention is obtained by compressing a mixture that comprises granulated metformin HCl. Granulated metformin HCl is commercially available at Vistin Pharma (Oslo, Norway) or can be manufactured by using Avicel® DG as disclosed in EP 2 938 362 B1.

A preferred extended-release tablet core of the present invention comprises at least one vitamin B12 depleting drug, at least one controlled release agent, at least one diluent, at least one lubricant and optionally at least one source of calcium ions, and wherein said source of calcium ions is preferably a calcium salt, and wherein said calcium salt is preferably selected from the group consisting of calcium phosphate, calcium carbonate and calcium citrate, and wherein said calcium salt is more preferably selected from the group consisting of anhydrous calcium citrate, tricalcium dicitrate tetrahydrate, anhydrous monocalcium phosphate, anhydrous dicalcium phosphate, anhydrous tricalcium phosphate and calcium carbonate.

In case the at least one vitamin B12 depleting drug is metformin or a pharmaceutically acceptable salt thereof, the source of calcium ions is preferably calcium citrate, more preferably anhydrous calcium citrate, tricalcium dicitrate tetrahydrate or a mixture thereof. This does not exclude the possibility of adding—in addition to calcium citrate—a further source of calcium ions. By way of example, if metformin HCl is granulated with Avicel® DG, the extended-release tablet core of the present invention comprises metformin HCl, at least one controlled release agent, at least one lubricant, at least one diluent (being preferably microcrystalline cellulose), dibasic calcium phosphate anhydrous and calcium citrate, wherein said calcium citrate is preferably anhydrous calcium citrate, tricalcium dicitrate tetrahydrate or a mixture thereof.

In case the at least one vitamin B12 depleting drug is not metformin, the extended-release tablet core of the present invention may but does not need to comprise a source of calcium ions. Whereas calcium ions override metformin induced malabsorption of vitamin B12, calcium ions may or may not override malabsorption induced by any other vitamin B12 depleting drug. However, even if they do not override malabsorption, the other vitamin B12 depleting drug may also benefit from granulation with an excipient such as Avicel® DG. Thus, the extended-release tablet core of the invention may comprise a calcium salt even if the vitamin B12 depleting drug is not metformin.

The release rate of the extended-release tablet core of the present invention is controlled by the pharmaceutically acceptable excipients and in particular by the controlled release agent but—surprisingly—not by calcium citrate. Thus, calcium citrate is the preferred source of calcium ions.

In a preferred embodiment, after oral intake of the solid oral dosage form of the invention, less than 40 weight-%, more preferably less than 30 weight-%, even more preferably than 20 weight-% and most preferably less than 10 weight-% of the vitamin B12 depleting drug is released in the stomach of a patient in fasted condition.

In the intestine, metformin competes with calcium ions for the mucosal cell membrane. Therefore, a higher amount of calcium ions increases absorption of vitamin B12 more than a lower amount of calcium ions (unless saturation is achieved). A preferred extended-release tablet core of the present invention comprises from 0.0001 to 0.012 mol Ca²⁺, preferably from 0.0003 to 0.006 mol Ca²⁺ and most from 0.0005 to 0.001 mol Ca²⁺

One problem underlying the present invention is the provision of an improved version of the commercially available combination drug SYNJARDY® XR. In the best-case scenario, the package leaflet of such improved version will no longer need the warning that long-term treatment may cause vitamin B12 deficiency. SYNJARDY® XR comprises 1000 mg metformin HCl. Therefore, a preferred extended-release tablet core of the present invention comprises 1000 mg metformin HCl and from 0.0001 to 0.012 mol Ca²⁺, preferably from 0.0003 to 0.006 mol Ca²⁺ and most from 0.0005 to 0.001 mol Ca²⁺.

The solid oral dosage form of the invention comprises the extended-release core of the invention, preferably the extended-release tablet core of the invention. Therefore, a preferred embodiment of the invention relates to a solid oral dosage form comprising an extended-release tablet core that is coated with an immediate-release coating,
wherein said extended-release tablet core comprises from 500 mg to 1000 mg metformin HCl, from 50 mg to 300 mg calcium citrate, at least one controlled release agent, at least one diluent and at least one lubricant, and
wherein said calcium citrate is preferably anhydrous calcium citrate, tricalcium dicitrate tetrahydrate or a mixture thereof, and
wherein said immediate-release coating comprises vitamin B12 or a spray dried formulation thereof, and
wherein said extended-release tablet core comprises 1000 mg metformin HCl.

Coating(s)

The solid oral dosage form of the invention comprises at least one coating. In case it comprises one coating only, said coating is the herein described immediate-release coating. For releasing vitamin B12 into the patient's stomach, one immediate-release coating is sufficient. Nevertheless, the solid oral dosage form of the invention may comprise multiple (e.g. two or three) immediate-release coatings as herein described. In such less preferred embodiment, each of the immediate-release coatings release vitamin B12 into the patient's stomach.

In addition to the at least one immediate-release coating, the solid oral dosage form of the invention comprises optionally the herein described protective coating. Whereas one protective coating is sufficient to protect vitamin B12 as contained in the immediate-release coating from light, the solid oral dosage form of the invention may comprise multiple (e.g. two or three) protective coatings as herein described. Preferably, the solid oral dosage form of the invention comprises one protective coating which, upon oral administration of the solid oral dosage form, gets in contact with the patient's saliva.

Despite of the protective coating(s), the vitamin B12 is promptly released into the stomach such that after oral intake of the solid oral dosage form, preferably at least 70 weight-%, more preferably at least 80 weight-%, even more preferably at least 85 weight-% and most preferably at least 90 weight-% of vitamin B12 is released in the stomach of a patient in fasted condition, based on the total weight of vitamin B12 in the solid oral dosage form. Thereby, the indicated weight-% related to the B12 vitamer cyanocobalamin. Thus, if the immediate-release coating comprises a spray dried formulation of vitamin B12, the indicated weight-% related to the total amount of cyanocobalamin in the spray-dried formulation of vitamin B12.

Preferably, the solid oral dosage form of the invention comprises one immediate-release coating and one protective coating, wherein said immediate-release coating comprises vitamin B12 or a spray dried formulation thereof and wherein said protective coating protects the immediate-release coating's vitamin B12 from light. To do so, the protective coating comprises preferably at least one light-protection agent such as titanium dioxide.

In case the immediate-release coating comprises a spray dried formulation of vitamin B12, said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12.

As for safety, “Tolerable Upper Intake Levels” (known as ULs) are set for vitamins and minerals when evidence is sufficient. In the case of vitamin B12, there is no UL, as there is no human data for adverse effects from high doses. Therefore, the total amount of vitamin B12 in the immediate-release coating (or if there is more than one in the immediate-release coatings) is not particularly limited. However, a preferred embodiment of the invention relates to a solid oral dosage form comprising an extended-release tablet core that is coated with an immediate-release coating,
wherein said extended-release tablet core comprises at least one vitamin B12 depleting drug, and
wherein said immediate-release coating comprises from 1 μg to 10 μg cyanocobalamin, more preferably from 1 μg to 6 μg cyanocobalamin and most preferably from 1 μg to 4 μg cyanocobalamin. Thereby, said cyanocobalamin may be added as crystalline vitamin B12, as a spray dried formulation of vitamin B12 or as a mixture thereof.

Optionally, the immediate-release coating and/or the protective coating may comprise at least one further active pharmaceutical ingredient (API). If the optional further API is present, it is preferably comprised in the immediate-release coating.

In case the at least one vitamin B12 depleting drug is metformin HCl, the optional further API is preferably empagliflozin or a pharmaceutically acceptable salt thereof. A preferred embodiment of the invention relates to a solid oral dosage form comprising an extended-release tablet core that is coated with an immediate-release coating,
wherein said extended-release tablet core comprises 1000 mg metformin HCl, optionally at least one source of calcium ions and at least one pharmaceutically acceptable excipient, and
wherein said immediate-release coating comprises from 1 μg to μg cyanocobalamin, more preferably from 1 μg to 6 μg cyanocobalamin and most preferably from 1 μg to 4 μg cyanocobalamin, and
wherein said immediate-release coating comprises from 5 mg to 25 mg empagliflozin, and wherein said immediate-release coating comprises 5 mg empagliflozin or 10 mg empagliflozin or 12.5 mg empagliflozin or 25 mg empagliflozin, and
wherein said immediate-release coating is at least partially covered by a protective coating, and
wherein the extended-release tablet core of said solid oral dosage form comprises optionally at least one calcium salt, and wherein said calcium salt is preferably calcium citrate, and wherein said calcium citrate is preferably anhydrous calcium citrate or tricalcium dicitrate tetrahydrate.

Method of Manufacturing

The present invention also relates to a method of manufacturing a solid oral dosage form which comprises an extended-release core and at least one vitamin B12 depleting drug, wherein said extended-release core is provided with an immediate-release coating that comprises vitamin B12 or a spray dried formulation thereof. Thereby, said extended-release core is preferably an extended-release tablet core.

In a preferred embodiment, the method of the invention comprises the steps:
a) providing an extended-release tablet core which comprises at least one vitamin B12 depleting drug, optionally at least one source of calcium ions and at least one pharmaceutically acceptable excipient, and
b) providing the extended-release tablet core of step a) with an immediate-release coating that comprises vitamin B12 or spray dried formulation thereof,
   wherein said at least one vitamin B12 depleting drug is metformin or a pharmaceutically acceptable salt thereof, and wherein said at least one vitamin B12 depleting drug is more preferably metformin HCl, and
   wherein said at least one source of calcium ions is preferably a calcium salt, and wherein said calcium salt is preferably selected from the group consisting of calcium phosphate, calcium carbonate and calcium citrate, and wherein said calcium salt is more preferably selected from the group consisting of anhydrous calcium citrate, tricalcium dicitrate tetrahydrate, anhydrous monocalcium phosphate, anhydrous dicalcium phosphate, anhydrous tricalcium phosphate and calcium carbonate.

Step a) comprises preferably the step of dry granulation of at least one vitamin B12 depleting drug with at least one calcium salt and optionally microcrystalline cellulose, wherein said calcium salt is preferably calcium phosphate, and wherein said calcium salt is most preferably anhydrous dicalcium phosphate.

Step b) is preferably done by spraying a liquid immediate-release coating onto the extended-release tablet core of step a). Said liquid immediate-release coating is preferably obtained by mixing vitamin B12 or spray dried formulation thereof with solvent (e.g. ethanol) and at least one pharmaceutically acceptable binder.

Spray dried formulations of vitamin B12 protect vitamin B12 from light. Therefore, the spray dried formulations of vitamin B12 should remain intact/undissolved in the liquid immediate-release coating. This can be achieved by choosing the solvent in a suitable manner. A preferred solvent is ethanol as both, vitamin B12 and commercially available spray dried formulations of vitamin B12 are hardly or not at all soluble in ethanol. Due to the red color of vitamin B12, the extended-release tablet core may have red spots that, before being covered with the optional protective coating, may be visible to the naked eye.

The liquid immediate-release coating may also comprise a further active pharmaceutical ingredient being preferably empagliflozin or a pharmaceutically acceptable salt thereof. Therefore, the method of the invention comprises preferably the steps:
a) providing an extended-release tablet core which comprises metformin HCl, at least one calcium salt and at least one pharmaceutically acceptable excipient, and
b) providing the extended-release tablet core of step a) with an immediate-release coating that comprises empagliflozin and vitamin B12 or spray dried formulation thereof,
   wherein said calcium salt is preferably calcium citrate, and wherein said calcium citrate is preferably anhydrous calcium citrate or tricalcium dicitrate tetrahydrate.

The present invention also relates to the use of a spray dried formulation of vitamin B12 for manufacturing an immediate-release coating, wherein said immediate-release coating covers at least partially an extended-release core which comprises at least one vitamin B12 depleting drug, and wherein said extended-release core is preferably an extended-release tablet core.

Use of Solid Oral Dosage Form

The present invention also relates to the herein described solid oral dosage form for use as a medicament. The herein described solid oral dosage form comprises preferably metformin HCl. Thus, one embodiment of the invention relates to a solid oral dosage form comprising an extended-release tablet core that is coated with an immediate-release coating,
wherein said extended-release tablet core comprises metformin HCl, optionally at least one source of calcium ions and at least one pharmaceutically acceptable excipient, and
wherein said immediate-release coating comprises vitamin B12 or a spray dried formulation thereof, for use in the treatment of a patient who is in need of metformin.

A patient suffering from diabetes may be in need of metformin. Therefore, the present invention also relates to the herein described solid oral dosage form for use in the treatment of diabetes. A preferred embodiment of the invention relates to a solid oral dosage form comprising an extended-release tablet core that is coated with an immediate-release coating,
wherein said extended-release tablet core comprises metformin HCl, optionally at least one source of calcium ions and at least one pharmaceutically acceptable excipient, and
wherein said immediate-release coating comprises empagliflozin and vitamin B12 or a spray dried formulation thereof. for use in the treatment of diabetes.

The present invention also relates to a method for the treatment or prevention of drug induced vitamin B12 deficiency, said method comprising the step of administering the herein described solid oral dosage form. In a preferred embodiment, the invention relates to a method for the treatment or prevention of metformin induced vitamin B12 deficiency, said method comprising the step of administering the herein described solid oral dosage form.

Long-term treatment with metformin has been associated with a decrease in serum vitamin B12 levels which may cause peripheral neuropathy. Therefore, the present invention also relates to a method for the prevention of metformin induced peripheral neuropathy, said method comprising the step of administering the herein solid oral dosage form. An alternative embodiment relates to a solid oral dosage form as herein described for use in the prevention of metformin induced peripheral neuropathy.

### FIGURES

FIG. 1 shows the dissolution profiles in Fasted State Simulated Gastric Fluid (FaSSGF) of uncoated tablet cores which comprise metformin HCl. Two kinds of cores were tested: with and without calcium salt. The composition of the two kinds of cores is given in Tables 2a and 2b.

FIG. 2 shows the dissolution profiles in Fasted State Simulated Intestinal Fluid (FaSSIF) of uncoated tablet cores which comprise metformin HCl. The same two types of cores were tested as in the experiment shown in FIG. 1.

FIGS. 3a, 3b and 3c relate to the dissolution profiles of the coated tablet cores. The coating comprises a source of vitamin B12. Two kinds of sources of vitamin B12 were tested: crystalline vitamin B12 and a spray dried formulation of vitamin B12. Thus, two kinds of coated cores were tested. The composition of the two kinds of coating is given in Tables 5a and 5b.

FIG. 3a shows the dissolution profiles of metformin and empagliflozin in FaSSGF in regard of crystalline vitamin B12. FIG. 3b shows the dissolution profiles of metformin and empagliflozin in FaSSGF in regard of a spray dried formulation of vitamin B12. FIG. 3c shows a comparison of the empagliflozin dissolution from the two kinds of coating in FaSSGF.

FIGS. 4a, 4b and 4c relate to similar experiments as shown in FIGS. 3a, 3b and 3c. For those experiments, however, the dissolution profiles were measured in FaSSIF instead of FaSSGF.

FIG. 4a shows the dissolution profiles of metformin and empagliflozin in FaSSIF in regard of crystalline vitamin B12. FIG. 4b shows the dissolution profiles of metformin and empagliflozin in FaSSIF in regard of a spray dried formulation of vitamin B12. FIG. 4c shows a comparison of the metformin dissolution from the two kinds of coating in FaSSIF.

### EXAMPLES

### Example 1 (Selection of the Preferred Source of Ionic Calcium)

In Example 1, the solubility of four different calcium salts was analysed in simulated intestinal fluid via determination of the Ca²⁺ ions content by ICP-OES (inductively coupled plasma optical emission spectrometry).

The calcium salts were:
calcium carbonate (95 MD available at Particle Dynamics),
dicalciumphosphat anhydrous (DiCafos A150, available at Budenheim),
tricalcium dicitrate tetrahydrate (available at Merck)
anhydrous calcium citrate (available at Gadot).

The solubilization medium was:
SIF (simulated intestinal fluid, pH=6.8, prepared according to Ph. Eur.)

The dissolution medium was heated to 37° C. and the analyses were performed at this temperature. During the analyses, the salts were added in access to the solubilization media and left to mix for 24 h. Afterwards, the solutions were filtered, and the precipitates were investigated using ATR-IR analysis to confirm the presence of the starting material (e.g. respective Ca salt). Attenuated total reflection (ATR) is a sampling technique used in conjunction with infrared spectroscopy which enables samples to be examined directly in the solid or liquid state without further preparation. The filtered solutions were analysed for Ca²⁺ ions content. The solubility results that were obtained are shown in the TABLE 1.

TABLE 1

| | SIF | | | | |
|---|---|---|---|---|---|
| | | solubility | | Ca²⁺ | |
| | solubility | mg/ml | | ions | Ca²⁺ ions |
| | mg/ml | average | pH | mg/ml | mg/ml, average |
| Ca carbonate | 0.058 | 0.06 | 7.5 | 0.023 | 0.024 |
| | 0.060 | | 7.5 | 0.024 | |
| dicalcium | 0.093 | 0.09 | 6.7 | 0.027 | 0.028 |
| phosphate | 0.094 | | 6.6 | 0.028 | |
| anhydrous | | | | | |
| tricalcium dicitrate | 2.130 | 2.11 | 6.1 | 0.450 | 0.445 |
| tetrahydrate | 2.090 | | 6.1 | 0.440 | |
| anhydrous calcium | 1.920 | 1.91 | 6.1 | 0.460 | 0.460 |
| citrate | 1.890 | | 6.1 | 0.460 | |

For in vivo absorption of vitamin B12, a vitamin B12-IF complex needs to be formed which then binds to enterocyte receptors in the ileum. For this process, calcium ions should be present in the patient's ileum.

Because the tablet core of the invention is an extended-release tablet core, it reaches the patient's intestine partially or fully undissolved. If the core comprises a calcium salt, said calcium salt reaches the patient's intestine also partially or fully undissolved. Calcium salts which remain undissolved in the ileum do not significantly increase absorption of vitamin B12. Thus, any calcium salt comprised in an extended-release core should be well soluble in intestinal fluid.

Example 1 shows that calcium citrate salts are well soluble in intestinal fluid. Therefore, calcium citrate salts such as tricalcium dicitrate tetrahydrate and anhydrous calcium citrate are particularly suitable to be included in the extended-release tablet core of the invention.

### Example 2 (Preparation of Uncoated Extended-Release Metformin Cores, with and without Calcium Salt)

For the tabletting mixture of the metformin cores, the individual components listed in Tables 2a and 2b, except magnesium stearate and glyceryl behenate (outer phase), were poured into a 2.5 L powder glass bottle and blended for 16 minutes at 32 rpm using a Turbula 3D mixer/blender (WAB plc. Muttenz, Switzerland). Subsequently magnesium stearate and glyceryl behenate were sieved through a 500 micron mesh sized sieve and added to the previous blend. After an additional blending of 4 minutes at 32 rpm this final tabletting mixture was transferred to the XP1 eccentric press from Korsch plc. (Berlin, Germany).

The only difference of the composition in case of the tablet cores without calcium citrate, the 14.5 wt.-% Ca citrate were compensated (replaced) with the addition of more Prosolv SMCC 90 (to 20.4 wt.-%, cf. TABLE 2b).

TABLE 2a shows the composition in case of the tablet cores with calcium citrate.

For all cores, a mean compression force of 31.52±1.16 kN and a tabletting speed of 10 tablets per minute was used.

TABLE 2a
Tablet core composition
(with tricalcium dicitrate tetrahydrate)

| | Weight | Weight |
|---|---|---|
| Component name/Supplier | (mg) | (wt.-%) |
| Metformin granules (92.6 wt.-%)/ | 863.9 | 57.6 |
| Vistin Pharma (Oslo, Norway) | | |
| Methocel K100M Premium CR/Colorcon | 300.0 | 20.0 |
| inc. (Dartford Kent, United Kingdom) | | |
| Prosolv SMCC 90/JRS Pharma ltd. & | 88.7 | 5.9 |
| Co. KG (Rosenberg, Germany) | | |
| Tricalcium dicitrate tetrahydrate/Merck | 217.4 | 14.5 |
| (Darmstadt, Germany) | | |
| Magnesium stearate/Hanseler plc. | 15.0 | 1.0 |
| (Herisau, Switzerland) | | |
| Glyceryl behenate (Compritol 888 ATO) | 15.0 | 1.0 |
| Gattefosse plc. (Saint-Priest, France) | | |
| Sum | 1500.0 | 100.0 |

TABLE 2b
Tablet core composition
(without tricalcium dicitrate tetrahydrate)

| | Weight | Weight |
|---|---|---|
| Component name/Supplier | (mg) | (wt.-%) |
| Metformin granules (92.6 wt.-%)/Vistin Pharma | 863.9 | 57.6 |
| (Oslo, Norway) | | |
| Methocel K100M Premium CR/Colorcon inc. | 300.0 | 20.0 |
| (Dartford Kent, United Kingdom) | | |
| Prosolv SMCC 90/JRS Pharma ltd. & Co. KG | 306.1 | 20.4 |
| (Rosenberg, Germany) | | |
| Tricalcium dicitrate tetrahydrate/Merck | — | — |
| (Darmstadt, Germany) | | |
| Magnesium stearate/Hanseler plc. (Herisau, | 15.0 | 1.0 |
| Switzerland) | | |
| Glyceryl behenate (Compritol 888 ATO) | 15.0 | 1.0 |
| Gattefosse plc. (Saint-Priest, France) | | |
| Sum | 1500.0 | 100.0 |

The punches that were used had a diameter of 20 mm with two round flat-faced faceted sides. A tablet weight of 1.50 g was targeted, and the resulting core properties are listed in TABLE 3. Tablet weights were recorded on a PB 303-LDR Delta Range balance from Mettler Toledo Itd. (Greifensee, Switzerland). The breaking force and tablet height were measured with the tablet hardness tester TBH 220 TD from Erweka ltd. (Heusentamm, Germany). Friability testing was done according to USP <1216> (10 tablets 100 revolutions at 25 rpm) using the TA 120 friability tester from Erweka Itd. (Heusenstamm, Germany).

TABLE 3

| | without tricalcium | with tricalcium |
|---|---|---|
| core formulation | dicitrate tetrahydrate | dicitrate tetrahydrate |
| Uniformity of mass | 1.50 ± 0.002 | 1.50 ± 0.003 |
| [g] (n = 30) | | |
| Uniformity of height | 4.22 ± 0.01 | 4.25 ± 0.01 |
| [mm] (n = 30) | | |
| Mean breaking force | 150.20 ± 4.47 | 147.40 ± 3.50 |
| [N] (n = 10) | | |
| Friability [weight | 0.13 | 0.18 |
| loss %] (n = 10) | | |

### Example 3 (Dissolution Data of Uncoated Extended-Release Metformin Cores, with and without Calcium Salt)

In Example 3, the effect of calcium salt addition on the release of metformin HCl was tested.

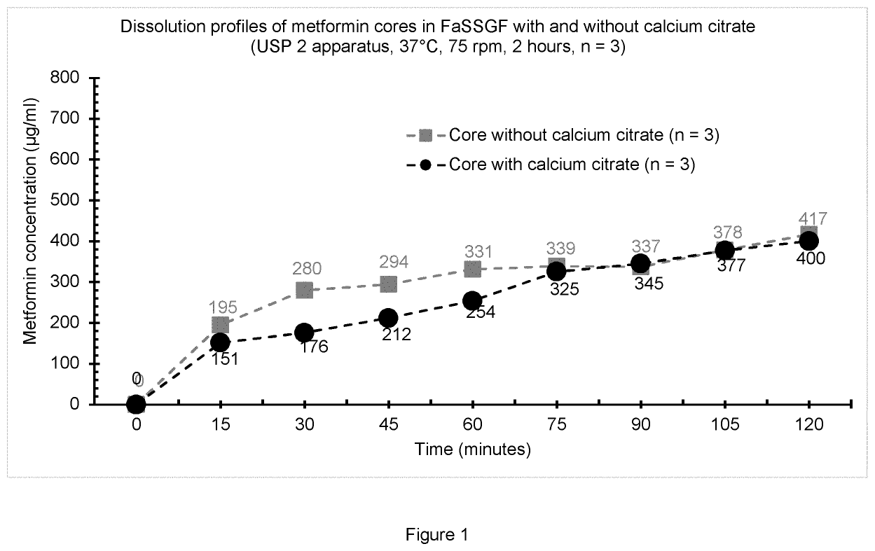

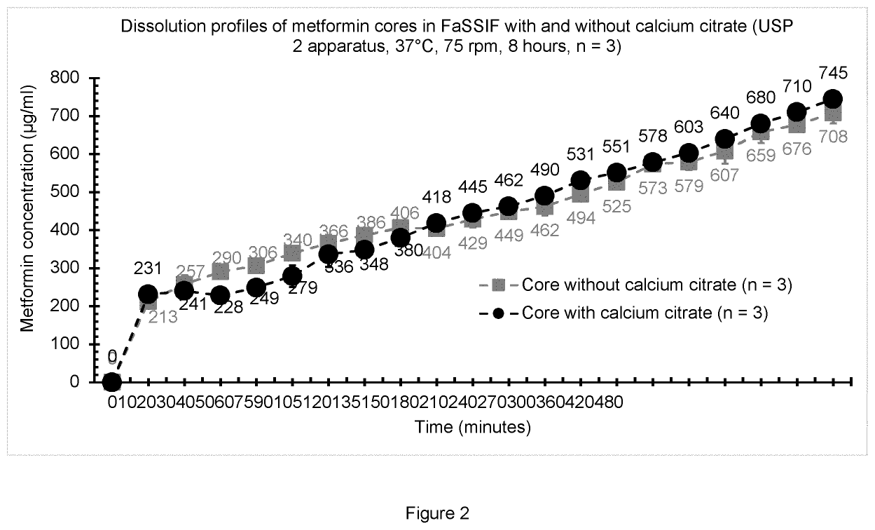

Dissolution testing was performed in triplicate on the USP 2 apparatus DT600 HH from Erweka Itd. (Heusenstamm, Germany). A paddle speed of 75 rpm and a temperature of 37° C. was used. The resulting dissolution profiles are shown in FIGS. 1 and 2. As dissolution media for each core 900 ml of Fasted State Simulated Gastric Fluid (FaSSGF) Fasted State Simulated Intestinal Fluid (FaSSIF) were used.

Preparation of the Bio-Simulating Media FaSSIF and FaSSGF:

A detailed overview of the compositions is depicted in TABLE 4. For 1 liter of the FaSSIF buffer, 0.420 g of sodium hydroxide pellets (Sigma-Aldrich Chemie plc.; Buchs, Switzerland), 3.954 g of monobasic sodium phosphate anhydrous (Sigma-Aldrich Chemie plc.; Buchs, Switzerland) and 6.186 g sodium chloride (Sigma-Aldrich Chemie plc.; Buchs, Switzerland) were dissolved in 1 liter purified water. The pH value was adjusted with 1M hydrochloric acid (Merck KGaA; Darmstadt, Germany). Finally, 2.240 g of the SIF powder (Biorelevant.com ltd.; London United Kingdom) were dissolved in a total volume of 1 L buffer. After 2 hours of stirring on a magnetic stirring plate, the FaSSIF was ready to use.

In 1 liter of the buffer for the bio-relevant media FaSSGF, 1.999 g sodium chloride (Sigma-Aldrich Chemie plc.; Buchs, Switzerland) were dissolved in liter purified water. The pH value was adjusted with 1M hydrochloric acid (Merck KGaA; Darmstadt, Germany). Finally, 0.0597 g of the SIF powder (Biorelevant.com Itd.; London, United Kingdom) were dissolved in a total volume of 1 L buffer. The purified water used for the media was treated with an arium Pro from Satorius Lab Instruments GmbH & Co. KG (Göttingen, Germany) and had a conductivity of ≤0.055 μS/cm at 25° C.

TABLE 4

| | FaSSIF composition | FaSSGF composition |
|---|---|---|
| Component | Concentration (mM) | |
| Taurocholate | 3 | 0.08 |
| Phospholipids | 0.75 | 0.02 |
| Sodium | 148 | 34 |
| Chloride | 106 | 59 |
| Phosphate | 29 | — |
| pH | 6.5 | 1.6 |

Samples of 2 ml were taken at different time points (see FIGS. 1 and 2) and the volume in the dissolution vessel was kept constant by immediate replacement with 2 ml of FaSSGF or FaSSIF. The samples were filtered through a Titan PTFE syringe filter (17 mm; 0.45 μm) from infochroma pic. (Goldau Switzerland) and immediately sealed in 2 ml brown HPLC glass vials (Wicom Germany ltd., Heppenheim, Germany).

HPLC Analysis:

Drug concentrations were measured in triplicates at room temperature (RT) using a reversed phase method on a HPLC 1200 series instrument from Agilent Technologies ltd. (Waldbronn, Germany) using an UV detector at 277 nm, 1 ml/min flowrate and 20 μL injection volume. The HPLC was equipped with a hydrophobic C18 column (ZORBAX Eclipse Plus, 5 μm, 2.1×150 mm) from Agilent Technologies. As mobile phase, 50% (v/v) methanol and 50% (v/v) phosphate buffer (pH 3.0) was used. For the phosphate buffer 6.8 g potassium phosphate monobasic was dissolved in 1 liter purified water and the pH was adjusted with phosphoric acid (85%) to 3.0. All chemicals used for the mobile phase were supplied from Sigma-Aldrich Chemie plc. (Buchs, Switzerland).

FIGS. 1 and 2 show the data obtained in Example 3.

### CONCLUSIONS

The data of Example 3 relates to uncoated tablet cores whereas the solid oral dosage form of the invention contains a tablet core that is covered with at least one immediate-release coating. Thus, after oral administration of the solid oral dosage form of the invention, the coating of the tablet core will first need to be dissolved. The data shown in FIG. 1 (FaSSGF) relates to a simulation of what will happen once the coating will have been dissolved. As shown in FIG. 1, in the stomach of the patient (i.e. in gastric fluid), only a limited amount of metformin will be released from the tablet core. FIG. 1 also shows that the addition of calcium citrated slightly delays the release of metformin in gastric juice. This is acceptable as the objective is the provision of an extended-release formulation of metformin.

Approximately 30-60 minutes after oral administration of the solid oral dosage form of the invention, the at least partially undissolved tablet core (without coating; coating has been dissolved in the stomach) will reach the patient's duodenum through the pylorus. The exact amount of time the core needs to pass from the stomach to the intestine depends on the patient's state (fed or fasted). The data shown in FIG. 2 (FaSSIF) relates to a simulation of what happens when the at least partially undissolved tablet core has reached the patient's intestine. The metformin cores with and without calcium show dissolution profiles of an extended release dosage form. After 8 hours in FaSSIF 89±3.5% of the metformin was released for the cores without calcium citrate and 93±1.3% of metformin for the cores with calcium citrate.

Thus, the addition of calcium citrate does not significantly influence the release of metformin in the intestine. This is an important aspect when seeking for line extension for an existing, approved extended-release formulation of metformin which allows once-daily dosing. It is expected that calcium citrate can be added to an existing, approved formulation without amending the release profile of metformin. In other words, when adding calcium citrate to an existing, approved formulation, a bioequivalent formulation is expected to be obtained.

### Example 4 (Coating of Cores Comprising Calcium Salt and Using Two Different Sources of Vitamin B12)

In Example 4, solid oral dosage forms according to a preferred embodiment of the invention were prepared.

First, cores identical to the ones of Example 2 were manufactured. The composition of the cores was identical to the composition shown in Table 2, i.e. the cores comprised metformin HCl and tricalcium dicitrate tetrahydrate. The thus obtained extended-release cores were then coated.

Coating of the extended-release cores was performed using a lab coater GC-1 from Glatt (Pratteln, Switzerland). Two different batches of coated tablets were produced. In the first batch, crystalline vitamin B12 was used as source of vitamin B12. In the second batch, a spray-dried formulation of vitamin B12 (0.1% WS, commercially available at DSM Nutritional Products, Switzerland) was used as source of vitamin B12.

In both cases, the tablet cores were coated with two coating layers. The first coating layer contained vitamin B12 (either crystalline vitamin B12 or spray dried formulation of vitamin B12) and empagliflozin. A second coating layer was then added on the top of the first coating layer as a protective layer. In the first batch, where crystalline vitamin B12 was used as source of vitamin B12, a rather thick protective layer was added because crystalline vitamin B12 is highly sensitive to light. In the second batch, where a spray dried formulation of vitamin B12 was used as source of vitamin 12, a rather thin protective layer was added because spray dried formulations of vitamin B12 are less light sensitive than crystalline vitamin B12.

TABLE 5a relates to the use of crystalline vitamin B12 as source of vitamin B12 (i.e. first batch) and shows the composition of both coating layers.

TABLE 5a

| | | |
|---|---|---|
| Ingredient | Amount (g) | Amount (wt.-%) |

Composition of the first coating layer

| | | |
|---|---|---|
| Eudragit E PO Ready-Mix | 80.0 | 10.14 |
| (contains TiO₂) | | |
| Empagliflozin | 13.2 | 1.67 |
| Vit.B12 (crystalline) | 0.01056 | 0.00134 |
| Ethanol abs. (0.789 g/ml) | ad 800 ml | 88.19 |

Composition of the second coating layer

| | | |
|---|---|---|
| Eudragit E PO Ready-Mix | 20.0 | 12.67 |
| (contains TiO₂) | | |
| Ethanol abs. (0.789 g/ml) | ad 200 ml | 87.33 |

TABLE 5b relates to the use of a spray dried formulation of vitamin B12 as source of vitamin B12 (i.e. second batch) and also shows the composition of both coating layers.

TABLE 5b

| | | |
|---|---|---|
| Ingredient | Amount (g) | Amount (wt.-%) |

Composition of the first coating layer

| | | |
|---|---|---|
| Eudragit E PO Ready-Mix | 80.0 | 10.14 |
| (contains TiO₂) | | |
| Empagliflozin | 13.2 | 1.67 |
| Vit.B12 (0.1% WS) | 10.56 | 1.34 |
| Ethanol abs. (0.789 g/ml) | ad 800 ml | 86.85 |

Composition of the second coating layer

| | | |
|---|---|---|
| Eudragit E PO Ready-Mix | 20.0 | 12.67 |
| (contains TiO₂) | | |
| Ethanol abs. (0.789 g/ml) | ad 200 m | 87.33 |

The first coating layer contains Eudragit E PO Ready-Mix from Evonik (Darmstadt, Germany). It is a ready to use mixture that comprises basic butylated methacrylate copolymer, talcum and titanium dioxide. Empagliflozin (BOC Sciences, Shirley N.Y., USA) was added in sufficient amount to reach the dosage of 25 mg/tablet. All solid ingredients were weighted and added to a glass beaker. About 10% of solid content in solvent was provided to obtain a well pumpable coating suspension. To the solids in a glass beaker ⅔ of the total amount EtOH abs. was added and stirred continuously for around 45 min with an IKA stirrer blade (Staufen, Germany) at a speed of around 200 rpm. Afterwards the rest of the EtOH abs. was added and mixed for 5 min to obtain a homogenous suspension.

The same process parameters were used for all coating processes; they are presented at the TABLE 6.

TABLE 6
Process parameter

| | |
|---|---|
| Inlet temperature (° C.) | 35 |
| Volume flow rate air (m³/h) | 30 |
| Rotation speed drum (rpm) | 5 |
| Spraying rate (mL/min) | 9 |
| Atomizing Air pressure (bar) | 1.0 |
| Pattern Air pressure (bar) | 1.0 |
| Number of coated tablets | 50 |
| per batch | |

Weight gain and time of coating for each of the tablet batches is presented in TABLE 7. For the batch where crystalline vitamin B12 was used as source of vitamin B12, coating time for second coating layer was increased to obtain a thicker protective layer. The targeted increase in thickness was confirmed by a higher weight gain.

TABLE 7

| | With | With |
|---|---|---|
| | Vit.B12 | Vit.B12 |
| | cryst. | 0.1% WS |
| First coating layer | | |
| Weight gain (wt.-%, based | 13.3 | 13.4 |
| on the weight of the tablet core) | | |
| Coating time (min) | 60 | 70 |
| Second coating layer | | |
| Weight gain (wt.-%, based on the weight of | 3.3 | 1.9 |
| the tablet core including the first coating layer) | 20 | 13 |
| Coating time (min) | | |

The results of the assay analyses are presented in the TABLE 8.

TABLE 8

| | Coated tablets with vit. | Coated tablets with vit. |
|---|---|---|
| Vitamin B12 type | B12 (0.1% WS) | B12 (crystalline) |
| Uniformity of mass | 1.740 ± 0.013 g (n = 19) | 1.770 ± 0.018 g (n = 24) |
| Mass gain including | 0.270 g | 0.240 g |
| second coating | | |

### Example 5 (Content Analysis of the Two Different Types of Solid Oral Dosage Forms Manufactured in Example 4)

The contents of vitamin B12, metformin and empagliflozin was measured in the solid oral dosage forms manufactured in Example 4.

Vitamin B12 Content Analysis:

The assay of vitamin B12 for the coated tablets was done also using a HPLC method. Vitamin B12 is a collective term for a group of vitamers. All vitamers using this method were determined as cyanocobalamin. Sample preparation was as follows: Four individual tablets were analysed. The individual vitamers are extracted from the sample using sodium acetate buffer and are transformed into the vitamer cyanocobalamin (R═—CN) with the aid of potassium cyanide. For purification, an aliquot of the extract is passed through an immunoaffinity column that contains specific antibodies that bind selectively to cyanocobalamin. The cyanocobalamin is then eluted from the column using methanol. The detection was done at the wave length of 361 nm.

Mean vit. B12 content for spray dried formulation of vitamin B12 of coated tablets is shown in Table 9.

TABLE 9

| | Tablets containing |
|---|---|
| | Vitamin B12 0.1% WS |
| Tablet no.: | n = 3 (μg/tablet) |
| 1 | 11.4 |
| 2 | 11.9 |
| 3 | 12.1 |
| Overall mean | 11.8 |
| Overall ± std. dev. | 0.38 |

Metformin and Empagliflozin Content Analysis:

Five tablets for each batch of the coated tablets (with the crystalline vit. B12 and with the vit. B12 0.1% WS titration) were analyzed regarding their API contents for metformin and empagliflozin. For this purpose, each tablet was dispersed in 250 ml of the HPLC mobile phase (50:50 v/v methanol:phosphate buffer pH 3.0) and stirred at 800 rpm (magnetic stirrer) for 48 hours in aluminum foil covered bottles. From each tablet dispersion, 4 samples were taken and diluted to 1:4 with the HPLC mobile phase to a total concentration of tablet per liter mobile phase. Prior to HPLC analysis, the diluted samples were mixed and filtered with Titan PTFE syringe filters (17 mm; 0.45 μm) from infochroma plc. (Goldau Switzerland). The resulting drug contents are shown in Tables 10a and 10b.

TABLE 10a relates to the use of crystalline vitamin B12 as source of vitamin B12 and shows mean metformin and empagliflozin concentrations.

TABLE 10a
Coated tablets with crystalline vitamin B12

| | Mean metformin | Mean empagliflozin |
|---|---|---|
| | concentration n = 4 | concentration n = 4 |
| Tablet no.: | (mg/tablet) | (mg/tablet) |
| 1 | 811.02 | 24.83 |
| 2 | 819.96 | 26.63 |
| 3 | 833.56 | 26.31 |
| 4 | 827.60 | 25.55 |
| 5 | 827.86 | 26.25 |
| Overall mean | 824.00 | 25.91 |
| Overall ± std. dev. | 8.72 | 0.72 |

TABLE 10b relates to the use of a spray-dried formulation of vitamin B12 as source of vitamin B12 and also shows mean metformin and empagliflozin concentrations.

TABLE 10b
Coated tablets with vitamin B12 titration (0.1% WS)

| | Mean metformin | Mean empagliflozin |
|---|---|---|
| | concentration n = 4 | concentration n = 4 |
| Tablet no.: | (mg/tablet) | (mg/tablet) |
| 1 | 854.03 | 25.48 |
| 2 | 834.08 | 24.97 |
| 3 | 831.49 | 26.59 |
| 4 | 820.86 | 25.30 |
| 5 | 832.01 | 24.56 |
| Overall mean | 834.49 | 26.38 |
| Overall ± std. dev. | 12.07 | 0.76 |

### Example 6 (Dissolution Test of the Two Different Types of Solid Oral Dosage Forms Provided in Example 4 in Gastric Fluid)

Upon coating, the dissolution profiles in FaSSGF of metformin, empagliflozin and Vitamin B12 from the coated tablets were analyzed in the same way as uncoated tablet cores described in example 3.

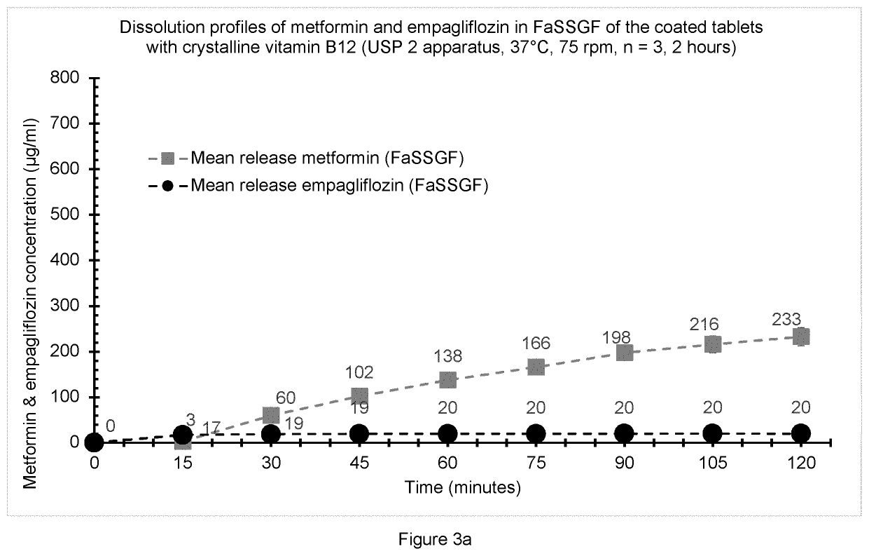

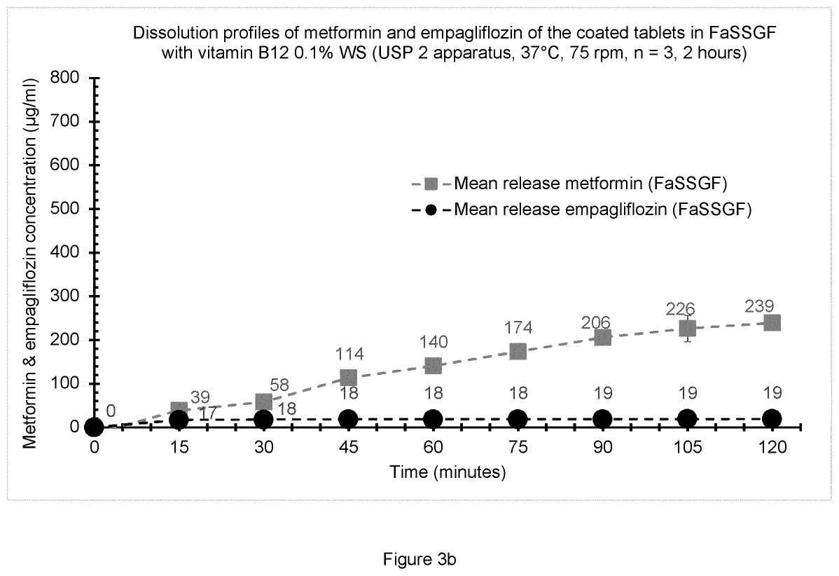

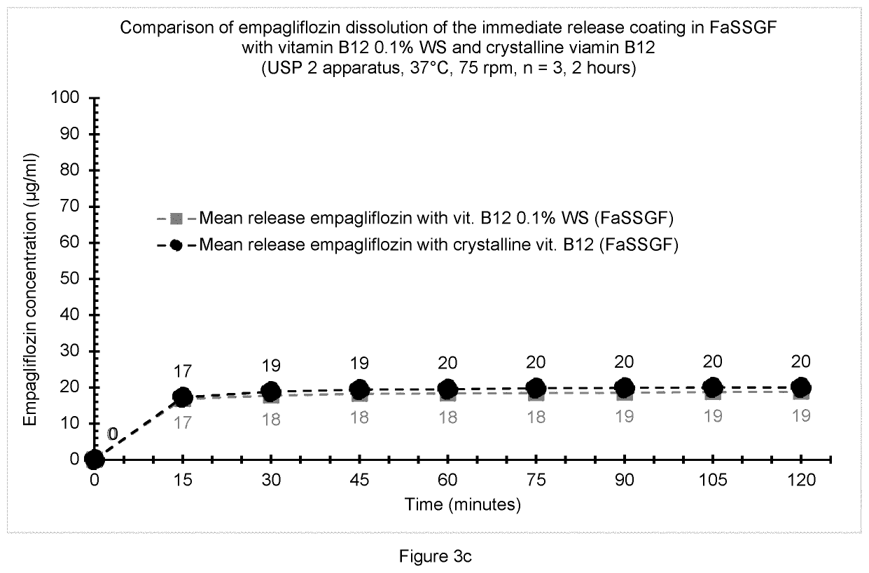

FIGS. 3a, 3b and 3c show the dissolution profiles of metformin and empagliflozin in FaSSGF.

FIG. 3a shows the dissolution profiles of metformin and empagliflozin in FaSSGF of the coated tablets with crystalline vitamin B12. FIG. 3b exhibits dissolution profiles of metformin and empagliflozin in FaSSGF of the coated tablets with spray dried formulation of vitamin B12 (0.1% WS). FIG. 3c shows a comparison of the empagliflozin dissolution from the immediate release coating in FaSSGF with vitamin B12 0.1% WS and crystalline vitamin B12.

There is no significant difference between the two batches in dissolution profiles when crystalline vitamin B12 or the spray dried formulation of vitamin B12 (WS 0.1%) is used in the coating.

After 30 minutes, the immediate release coating is completely dissolved in FaSSGF for both batches and after 15 minutes, empagliflozin is already dissolved up to 66.4±0.2%.

For both batches, less than 30% of the metformin is dissolved after 2 hours dissolution (37° C. and 75 rpm) in FaSSGF and after 1 hour only around 17%. It is expected that after this amount of time, the remains of the solid dosage form will have passed from the stomach (gastric fluid) through the pylorus into the duodenum (intestinal fluid).

### Example 7 (Dissolution Test of the Two Different Types of Solid Oral Dosage Forms Provided in Example 4 in Intestinal Fluid)

The dissolution profiles in FaSSIF of metformin, empagliflozin and Vitamin B12 from the coated tablets were analyzed in the same way as uncoated tablet cores described in example 3.

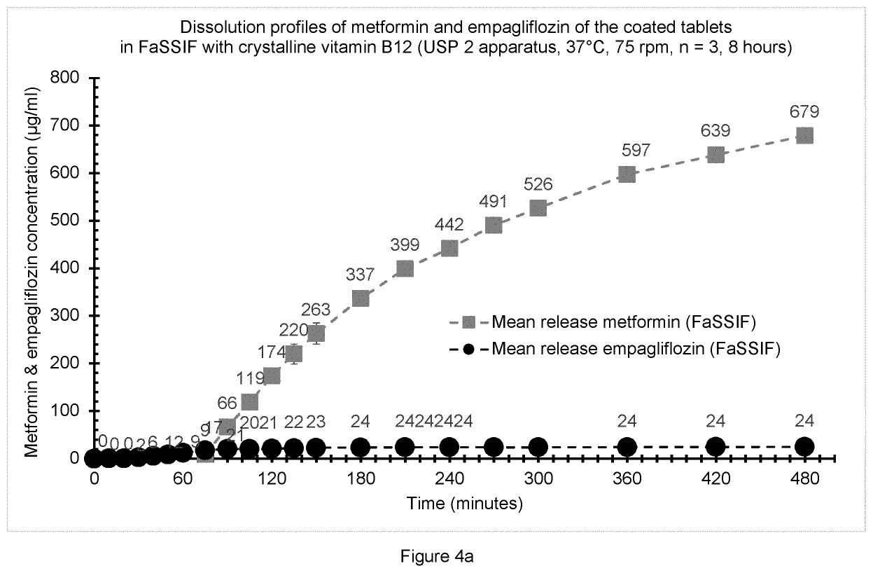

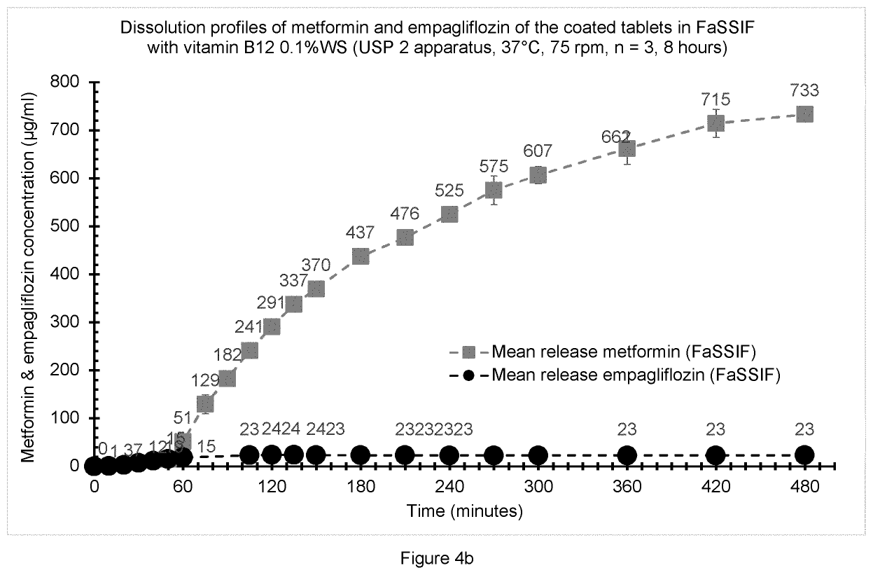

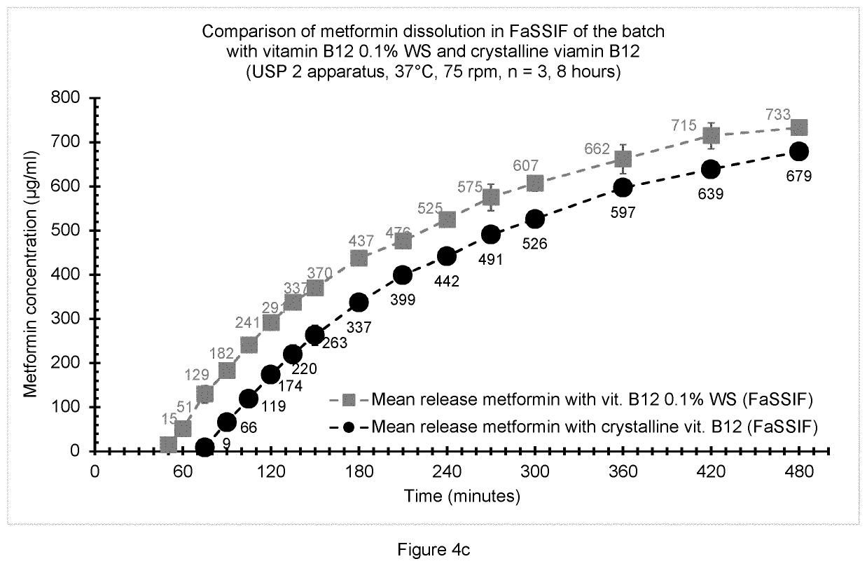

FIGS. 4a, 4b and 4c show the dissolution profiles of metformin and empagliflozin in FaSSIF.

FIG. 4a displays the dissolution profiles of metformin and empagliflozin in FaSSIF of the coated tablets with crystalline vitamin B12.

FIG. 4b depicts the dissolution profiles of metformin and empagliflozin in FaSSIF of the coated tablets with the spray dried formulation of vitamin B12 (0.1% WS).

FIG. 4c shows a comparison of the metformin dissolution in FaSSIF with vitamin B12 0.1% WS and crystalline vitamin B12.

In intestinal fluid, after dissolution of the two coating layers, an extended metformin release for both batches is clearly visible. After 8 hours, more than 80% of the metformin is released.

Even though an effect of the two coating layers can be seen in FIGS. 4a, 4b and 4c (i.e. lag-time), said effect will not be observed in vivo. After oral administration of the dosage form of the invention, the dosage form first arrives in the stomach where it is exposed to gastric juice. While being in the stomach, the two protective layers will be dissolved. At the time when the dosage form will be exposed do intestinal fluid, the two coating layers will be gone.

1. Solid oral dosage form comprising an extended-release tablet core that is coated with an immediate-release coating,
wherein said extended-release tablet core comprises at least one vitamin B12 depleting drug, and
wherein said immediate-release coating comprises vitamin B12 or a spray dried formulation thereof.

2. Solid oral dosage form according to claim 1,
wherein said immediate-release coating comprises a further active pharmaceutical ingredient, and wherein said immediate-release coating comprises preferably empagliflozin or a pharmaceutically acceptable salt thereof.

3. Solid oral dosage form according to claim 1,
wherein said at least one vitamin B12 depleting drug is metformin or a pharmaceutically acceptable salt thereof, and wherein said at least one vitamin B12 depleting drug is preferably metformin HCl.

4. Solid oral dosage form according to claim 3,
wherein said solid oral dosage form further comprises at least one source of calcium ions, and wherein said source of calcium ions is preferably a calcium salt, and wherein said calcium salt is preferably selected from the group consisting of calcium phosphate, calcium carbonate and calcium citrate, and wherein said calcium salt is more preferably selected from the group consisting of anhydrous calcium citrate, tricalcium dicitrate tetrahydrate, anhydrous monocalcium phosphate, anhydrous dicalcium phosphate, anhydrous tricalcium phosphate and calcium carbonate.

5. Solid oral dosage form according to claim 3,
wherein the extended-release tablet core of said solid oral dosage form comprises at least one calcium salt, and wherein said calcium salt is preferably calcium citrate, and wherein said calcium citrate is preferably anhydrous calcium citrate or tricalcium dicitrate tetrahydrate.

6. Solid pharmaceutical dosage form according to claim 1,
wherein said immediate-release coating comprises a spray dried formulation of vitamin B12, and wherein said spray dried formulation of vitamin B12 comprises preferably from 0.01 to 1 weight-%, more preferably from 0.05 to 0.5 weight-% and most preferably 0.1 weight-% cyanocobalamin, based on the total weight of the spray dried formulation of vitamin B12.

7. Solid pharmaceutical dosage form according to claim 1,
wherein said immediate-release coating is at least partially covered by a protective coating, and wherein said protective coating comprises preferably at least one light-protection agent such as titanium dioxide.

8. Solid oral dosage form according to claim 1,
wherein after oral intake of the solid oral dosage form, less than 30 weight-% of the vitamin B12 depleting drug is released in the stomach, based on the total weight of vitamin B12 depleting drug in the solid oral dosage form, and/or
wherein after oral intake of the solid oral dosage form, at least 70 weight-% of vitamin B12 is released in the stomach after oral intake of the solid oral dosage form, based on the total weight of vitamin B12 in the solid oral dosage form, and wherein said vitamin B12 is preferably cyanocobalamin.

9. Solid oral dosage form according to claim 1 for use as a medicament.

10. Solid oral dosage form according to claim 3 for use in the treatment or prevention of metformin induced vitamin B12 deficiency.

11. Method of manufacturing a solid oral dosage form which comprises an extended-release tablet core and at least one vitamin B12 depleting drug,
wherein said extended-release tablet core is provided with an immediate-release coating that comprises vitamin B12 or spray dried formulation thereof.

12. Method according to claim 10, said method comprising the step of dry granulation of at least one vitamin B12 depleting drug with at least one calcium salt and optionally microcrystalline cellulose,
wherein said calcium salt is preferably calcium phosphate, and wherein said calcium salt is most preferably anhydrous dicalcium phosphate.

13. Method according to claim 11, wherein said at least one vitamin B12 depleting drug is metformin or a pharmaceutically acceptable salt thereof, and wherein said at least one vitamin B12 depleting drug is preferably metformin HCl.

14. Method according to claim 11, wherein the solid oral dosage form is manufactured.

15. Use of a spray dried formulation of vitamin B12 for manufacturing an immediate-release coating,
wherein said immediate-release coating covers at least partially an extended-release tablet core which comprises at least one vitamin B12 depleting drug.
